(19) United States
(12) Patent Application Publication
(10) Pub. No.: US 2006/0094654 A1
(43) Pub. Date: May 4, 2006

(54) Neuroprotective complex for treatment of cerebral ischemia and injury

(76) Inventors: Nicolas G. Bazan, New Orleans, LA (US); Myron D. Ginsberg, Coconut Grove, FL (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 11/234,715

(22) Filed: Sep. 23, 2005

Related U.S. Application Data
(62) Division of application No. 10/763,698, filed on Jan. 23, 2004.

(51) Int. Cl. A61K 38/39; A61K 31/202

(52) U.S. Cl. 514/12; 514/560

(57) ABSTRACT

The invention provides a pharmaceutical composition useful in treating cerebral ischemia and cerebral injury. The pharmaceutical composition is also useful as a prophylactic treatment during surgical procedures wherein the potential for ischemic tissue damage is present. Also included in the invention is a method for preparing the pharmaceutical composition, as well as methods for treatment.

This is a divisional of co-pending application Ser. No. 10/763,698, filed Jan. 23, 2004.

### STATEMENT REGARDING FEDERALLY SPONSORED RESEARCH OR DEVELOPMENT

Not Applicable

### FIELD OF THE INVENTION

The present invention relates to a neuroprotective complex of human albumin and polyunsaturated fatty acid, particularly docosahexaenoic acid, that is useful in treating ischemic stroke, as well as other types of injuries, such as traumatic brain, eye and spinal cord injury, that may produce ischemic or traumatic tissue damage, and during surgical procedures such as carotid endarterectomy and coronary bypass surgery, where the potential for ischemic tissue damage is present.

### BACKGROUND OF THE INVENTION

Stroke is characterized by the sudden loss of circulation to an area of the brain, resulting in a corresponding loss of neurologic function. Also referred to as cerebrovascular accident or stroke syndrome, stroke is a nonspecific term encompassing a heterogeneous group of pathophysiologic causes, including thrombosis, embolism, and hemorrhage. Acute ischemic stroke refers to strokes caused by thrombosis or embolism and accounts for over 80% of all strokes.

The fundamental hypothesis in stroke research is that ischemia produces disability and death by initiating a cascade of cellular processes that eventually lead to neuronal death. The cascade begins rapidly after ischemia, when a thrombus or embolus from the heart, aorta, or carotid or vertebral arteries lodges in the intracranial circulation of the brain, blocking blood flow to the distal portion of the affected vessel. The processes involved in stroke injury at the cellular level are referred to as the ischemic cascade. Within seconds to minutes of the loss of glucose and oxygen delivery to neurons, the cellular ischemic cascade begins. This is a complex process that begins with cessation of the electrophysiologic function of the cells.

As the process continues, the cell metabolism changes from aerobic to anaerobic. With the depletion of ATP stores, membrane ion pumps fail, leading to increased intracellular concentration of sodium and calcium. The cell begins to experience injury from calcium-mediated cytotoxic reactions and release of excitatory neurotransmitters, specifically glutamate. These processes lead to activation of proteases, endonucleases, phospholipases, and nitric oxide synthase and formation of free radicals. The resultant neuronal and glial injury produces edema in the ensuing hours to days after stroke, causing further injury to the surrounding neuronal tissues.

An acute vascular occlusion produces heterogeneous regions of ischemia in the dependent vascular territory. The quantity of local blood flow comprises any residual flow in the major arterial source and collateral supply, if any. Regions of the brain without significant flow are referred to collectively as the core, and these cells are presumed to die within minutes. Zones of decreased or marginal perfusion are collectively called the ischemic penumbra. Tissue in the penumbra can remain viable for several hours, and pharmacologic interventions for preservation of neuronal tissue target the penumbra.

Drug therapies have been investigated, which, if administered after the onset of acute stroke, may potentially succeed in diminishing the extent of tissue damage and improving functional outcome. The proposed drug therapies are based on laboratory investigations of cerebral ischemia that have identified key biochemical and molecular mechanisms, including the central roles of excitotoxicity, tissue calcium overload, oxygen radicals, inflammatory mediators, and other factors, that contribute to the death of brain tissue.

Hemodiluting agents have been widely investigated as a potential therapy for ischemic stroke. The primary rationale for this approach is that cerebral blood flow varies inversely with hematocrit and whole-blood viscosity, and hemodilution has been shown to increase cerebral blood flow of both the normal and ischemic brain, either by decreasing blood viscosity or by vasodilation in response to diminished oxygen delivery. Albumin, an endogenous plasma protein, is commonly regarded as a hemodiluting agent. Importantly, albumin is an evolutionarily highly conserved molecule that subserves numerous vital physiological functions. Among these are fatty-acid transport, antioxidant function, maintenance of vascular endothelium, and oncotic activity. All of these functions are relevant to albumin's neuroprotective effect.

Several studies have reported a positive effect in reducing ischemic brain injury, including diminished brain edema and infarct volume, in rats with middle cerebral artery occlusion (MCAo) treated with high doses of albumin administered shortly after the onset of ischemia. Albumin has also been demonstrated to reduce the volume of contusion-injury in animals subjected to brain trauma. While albumin administration in humans has been found to be generally well tolerated, several adverse reactions may occur. When albumin is administered in high doses for the treatment of ischemia or other conditions, intravascular volume overload, congestive heart failure and pulmonary edema are the chief concerns. In rare circumstances, chills, fever, tachycardia, hypotension, urticaria, skin rash and nausea have been reported

As indicated above, high doses of human serum albumin, when administered intravenously within a therapeutic window extending up to four hours after the onset of MCAo, are highly neuroprotective reducing infarct volume and edema, thus improving neurological scores and protecting the ischemic penumbra. However, the effect of albumin therapy on local cerebral blood flow in areas that show histological neuroprotection is of lower magnitude than would be expected on the basis of its marked neuroprotectant effect. This suggests that other, non-hemodynamic mechanisms contribute to albumin-mediated neuroprotection.

In addition to albumin's neuroprotective characteristics, the protein is known to have several multifaceted intravascular effects. Albumin is a specific inhibitor of endothelial cell apoptosis. Several albumin-binding proteins have been identified on endothelial cells from many origins, including brain, that mediate its transcytosis and endocytosis. Albumin also constitutes a major antioxidant defense against oxidizing agents generated both by endogenous processes (such as neutrophil myeloperoxidase) and by exogenous compounds. Albumin also plays a crucial role in the transport of fatty acids and in the binding of metabolites and drugs. After considerable research, the inventors herein have discovered that albumin's role in the transport mechanism of fatty acids influences its neuroprotective effect.

### SUMMARY OF THE INVENTION

The inventors herein have discovered that the albumin-mediated systemic mobilization and supply of free fatty acids to the brain, which favors the replenishment of polyunsaturated fatty acids lost from cellular membranes during ischemia and/or serve as an alternate energy source, contributes to albumin neuroprotection. More specifically, MCAo selectively activates the transport of docosapentaenoic acid (22:5n-3) and docosahexaenoic acid (22:6n-3, DHA). Studies have suggested that polyunsaturated fatty acids may have therapeutic value for cerebral pathologies as they block neuronal death by inhibiting glutamatergic transmission. Surprisingly, however, it has been discovered that albumin loaded with polyunsaturated fatty acids, particularly docosahexaenoic acid (22:6n-3, DHA), produces high-grade histologic and neurologic protection at a dose considerably below that required when administering albumin alone.

Besides the neuroprotective function of the disclosed invention, the DHA-Albumin complex can also be used as a prophylactic treatment during surgical procedures wherein the potential for ischemic tissue damage is present. Ischemia/Reperfusion injury is a major cause of tissue damage and death that occurs when blood flow to an organ is interrupted and then later re-established, which can occur during major vascular surgery or in other situations. The potential for ischemic tissue damage may be reduced by the administration of DHA-Albumin complex, particularly with regard to surgical procedures involving large blood vessels, for example, procedures for treating thoracic and abdominal aortic aneurysms. Other procedures wherein ischemic tissue damage may be prevented by employing the treatments disclosed herein include coronary artery bypass grafting, coronary angioplasty, implantation of arterial stints, mesenteric and renal reconstruction, infrainguinal procedures, carotid endarterectomy, venous surgery, and major vascular trauma reconstructions.

### BRIEF DESCRIPTION OF THE DRAWINGS

FIG. 1 is a bar chart comparing total neuroscore of the identified treatment groups following MCAo. Repeated-measures ANOVA; *, different from all other groups, Dunnett's test; #, different from DHA-Alb 1.25 and from Alb 0.63 groups, Bonferroni test (p<0.05).

FIG. 2 is a bar chart comparing cortical infarct areas and volume of the identified treatment groups following MCAo. Repeated-measures ANOVA; *, different from saline group, Dunnett's test (p<0.05)

FIG. 3 is a bar chart comparing striatal infarct areas and volume of the identified treatment groups following MCAo. Repeated-measures ANOVA; *, different from saline group, Dunnett's test (p<0.05).

FIG. 4 is a bar chart comparing total infarct areas and volume of the identified treatment groups following MCAo. Repeated-measures ANOVA; *, different from saline group, Dunnett's test (p<0.05).

FIG. 5 is a bar chart comparing the neuroprotective effect of DHA-albumin complex with albumin expressed as a fraction of saline group. No significant differences between the two groups (Student t-tests).

### DETAILED DESCRIPTION OF THE INVENTION

The present invention relates to compositions comprising isolated complexes of polyunsaturated fatty acid (PUFA), particularly DHA, and albumin. The term “isolated” means the complex of PUFA and albumin is not in its natural state (e.g. not in the human body). The albumin used to obtain the complex can be either human serum albumin (hAlb) or recombinant albumin. The PUFA-Albumin complex may comprise more than one polyunsaturated fatty acid, as well as monounsaturated and saturated fatty acids. The complex is administered parenterally as soon as practicable after the onset of an acute brain insult resulting from ischemic stroke or traumatic injury and preferably within four hours of the insult. The complex can also be administered prophylatically during surgical procedures wherein the potential for ischemic tissue damage is present.

Pharmaceutical formulations for parenteral administration include, for instance, aqueous solutions of PUFA-Albumin complex or other appropriate suspensions. The pharmaceutical formulations are administered in a therapeutically effective dose, which refers to that amount of the complex that results in a reduction in the otherwise expected severity of ischemic or hemorrhagic tissue damage. The preferred dose for humans ranges between about 0.25 to about 2.5 grams (on an albumin weight basis) of PUFA-laden albumin per kilogram of body weight.

The present invention arose from a series of laboratory investigations wherein the effect of a DHA-Albumin complex was compared to treatment of albumin alone after an induced temporary MCAo. Animals were randomly assigned to 1 of 5 treatment groups: (1) Albumin, 1.25 g/kg (n=10); (2) DHA-Albumin, 1.25 g/kg (n=7); (3) Albumin, 0.63 g/kg (n=7); (4) DHA-Albumin, 0.63 g/kg (n=7); and (5) Normal saline (n=8).

The DHA-Albumin complex used in testing was prepared from the following protocol: Five vials containing 20 ml of human serum albumin (25%) were incubated with 4.0 mg DHA/g hAlb (molar ratio=0.2); incubation was performed in a shaking incubator at 37 degrees Centigrade for 30 minutes with vortex mixing every 5 min; aliquots (100 μl) from each vial were extracted and free fatty acids (FFA) were isolated by thin-layer chromatography (TLC), derivatized to fatty acid methyl esters (FAME) and analyzed by gas liquid chromatography (GLC); each vial was aliquoted in 5 ml samples and kept under nitrogen in a cold room for two months; and vials were gassed with nitrogen every week. There was no significant change in DHA and other polyunsaturated fatty acids loads on the hALb. The results show that the DHA-Albumin complex is stable with an expected product shelf life of at least about 4 to 6 weeks.

The DHA-Albumin complex in each of the five samples was analyzed to determine the amount of DHA loaded onto the albumin. The following tables illustrate the effect of the DHA incubation.

Fatty Acid Concentration (nmol/ml albumin)

| | Sample | Sample | Sample | Sample | Sample |
|---|---|---|---|---|---|
| Fatty Acid | 1 | 2 | 3 | 4 | 5 |
| 16:0 | 274.2 | 275.1 | 307.0 | 266.9 | 256.7 |
| 18:0 | 71.3 | 71.1 | 79.3 | 66.5 | 65.6 |
| 16:1 | 34.0 | 33.7 | 37.1 | 32.3 | 32.7 |
| 18:1 | 380.2 | 384.3 | 439.3 | 378.8 | 356.0 |
| 18:2n-6 | 230.2 | 191.0 | 247.9 | 213.2 | 201.2 |
| 20:4n-6 | 16.7 | 14.7 | 16.6 | 14.4 | 13.2 |
| 18:3n-3 | 13.7 | 12.9 | 14.9 | 13.7 | 12.1 |
| 20:5n-3 | 1.3 | 1.4 | 1.1 | 1.0 | 1.0 |
| 22:5n-3 | 8.0 | 8.4 | 9.4 | 8.0 | 6.4 |
| 22:6n-3 | 2005.6 | 2028.4 | 2219.8 | 2040.0 | 1589.3 |
| TOTAL: | 3035 | 3021 | 3372 | 3035 | 2534 |
| mg DHA/g hAlb | 2.64 | 2.67 | 2.92 | 2.68 | 2.09 |

Note that less than 4.0 mg DHA/g hAlb was incubated in Sample 5 resulting in a lower DHA load.

After preparing the DHA-Albumin complex, Male Sprague-Dawley rats (260-357 g) were anesthetized with halothane and nitrous oxide and subjected to up to 120 minutes of temporary MCAo by retrograde insertion of an intraluminal nylon suture coated with poly-L-lysine through the external carotid artery into the internal carotid artery and middle cerebral artery. Temperature probes were inserted in the rectum and the left temporalis muscle. Heating lamps were used to maintain rectal and temporalis muscle temperatures at 36 to 37 degrees Centigrade. In all rats, polyethylene catheters were introduced into the right femoral artery and vein for blood pressure recording, blood sampling, and drug infusion. Mean arterial blood pressure (MABP), plasma glucose, blood gases and hematocrit were continuously measured during the procedure.

Behavioral tests were performed in all 39 rats before MCAo, during occlusion at 60 minutes, and after treatment at 1 hour, 24 hours, 48 hours, and 72 hours. The battery consisted of two standardized tests used to evaluate various aspects of neurologic function: (1) the postural reflex test, which is used to examine upper body posture while the animal is suspended by the tail; and (2) the forelimb placing test, to examine sensorimotor integration in forelimb placing responses to visual, tactile and proprioceptive stimuli. Neurological function was graded on a scale of 0-12 (normal score=0, maximal score=12).

The drug (DHA-albumin, 1.25 or 0.63 g/kg; human albumin, 25% solution, 1.25 or 0.63 g/kg; or normal saline) was administered intravenously at the time of reperfusion, i.e., 2 hours from the onset of MCAo. The animals were allowed to survive for three days. Brains were then perfusion-fixed with a mixture of 40% formaldehyde, glacial acetic acid and methanol (FAM, 1:1:8 by volume), and infarct volumes and brain swelling were determined at 9 coronal levels throughout the brain. Repeated-measures ANOVA with post-hoc Bonferroni tests were used to assess infarct areas. Bonferroni-corrected Student t-tests were used in the non-repeated-measures comparisons. P<0.05 was regarded as significant.

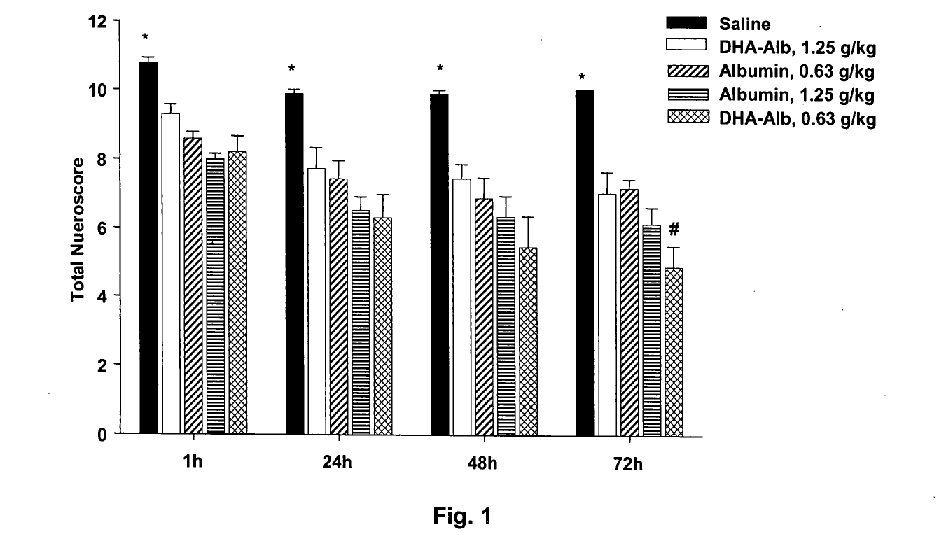

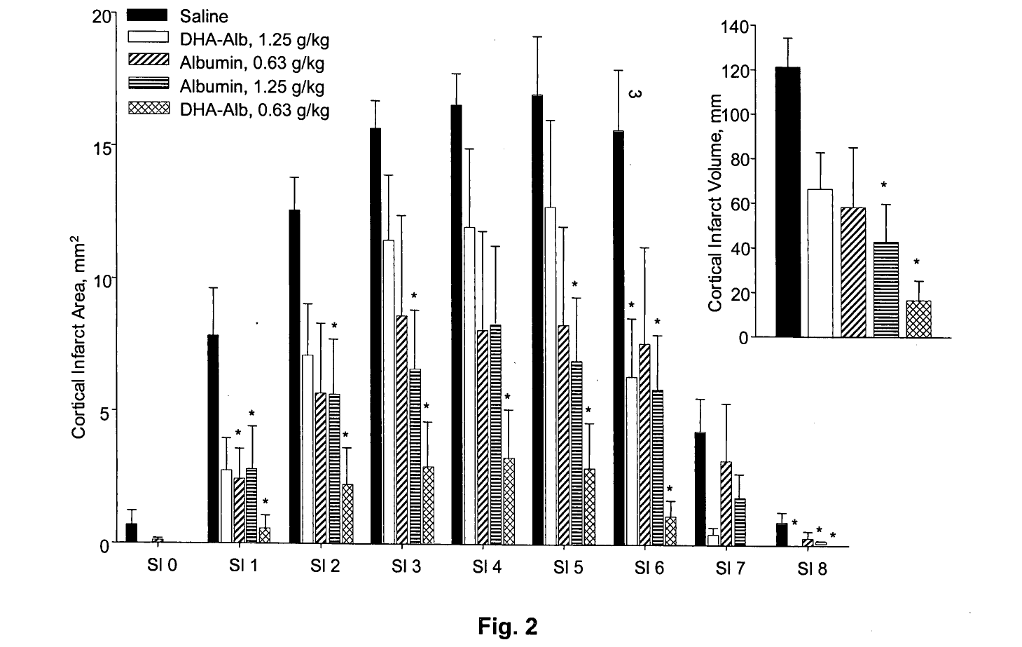

Physiological variables were stable and showed no significant differences among treatment groups. As can be seen in FIG. 1, while all treatments improved total neuroscore compared to saline, DHA-Albumin, 0.63 g/kg treatment reduced the 72 hr neuroscore significantly more than Albumin, 1.25 g/kg. FIG. 2 illustrates that DHA-Albumin, 0.63 g/kg treatment reduced cortical infarct areas at multiple coronal levels and it markedly reduced integrated cortical infarct volume by approximately 85%. While Albumin, 1.25 g/kg treatment also showed high-grade neuroprotection, the DHA-Albumin, 0.63 g/kg treatment group tended to be more highly protective (p=NS, i.e., >0.05).

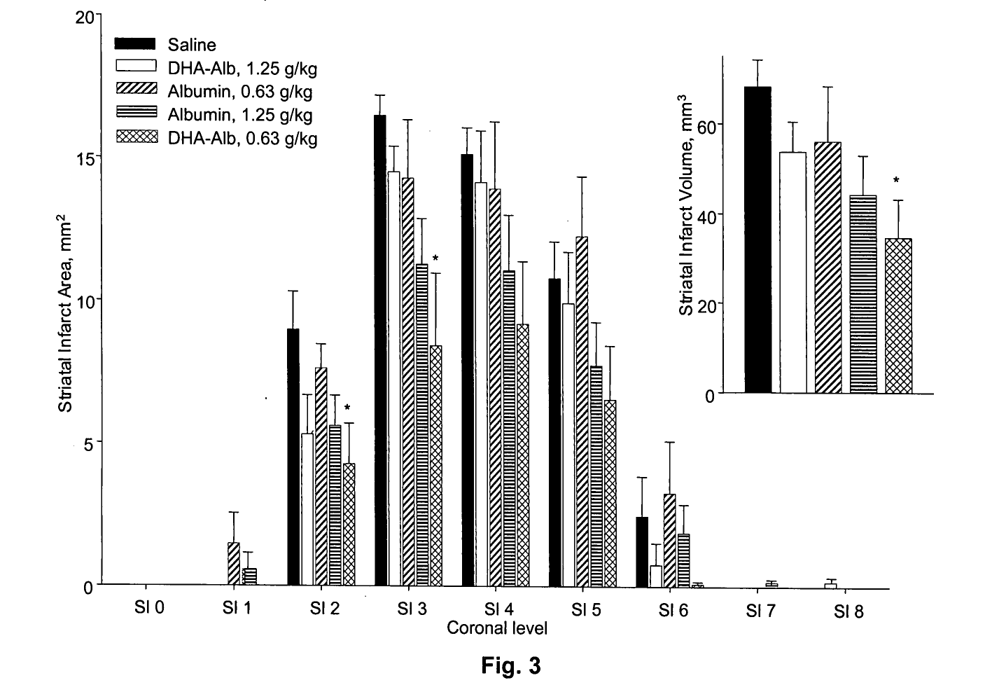

As shown in FIG. 3, the size of the subcortical infarct was reduced at 2 levels by DHA-Albumin, 0.63 g/kg treatment, and integrated striatal infarct volume was significantly reduced. Albumin alone was not able to achieve this effect.

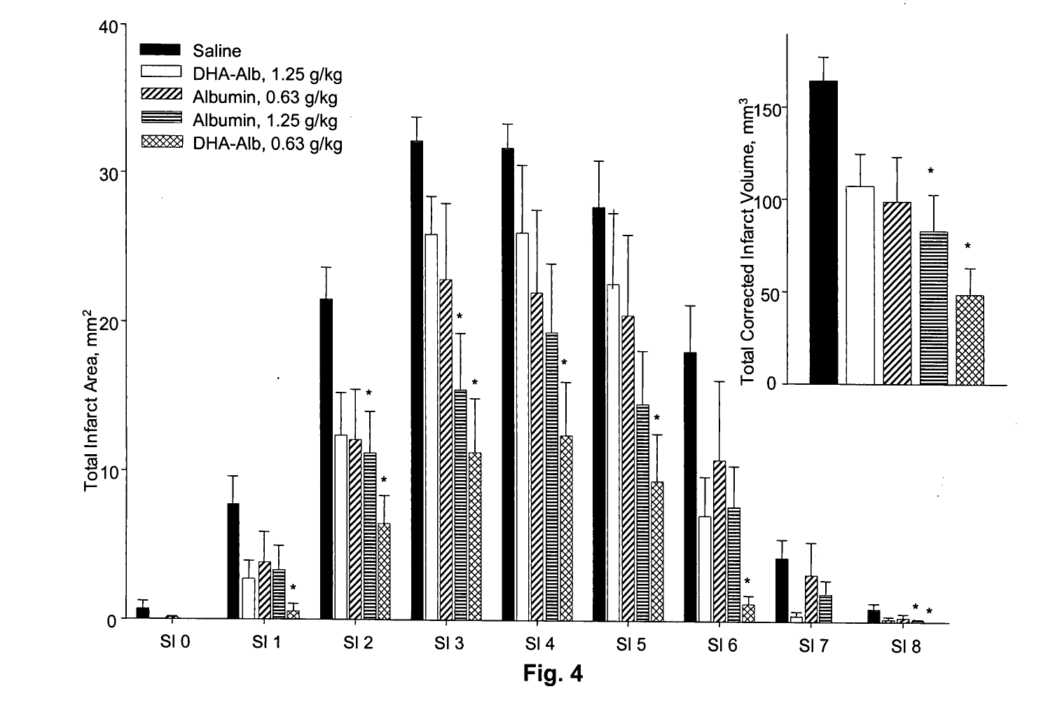

In FIG. 4, the total (cortical+subcortical) infarct was protected by DHA-Albumin, 0.63 g/kg treatment at multiple coronal levels, and the total (edema-corrected) infarct volume was reduced by approximately 70%. This degree of neuroprotection tended to be greater than the protection conferred by Albumin, 1.25 g/kg treatment, although this comparison was not statistically significant.

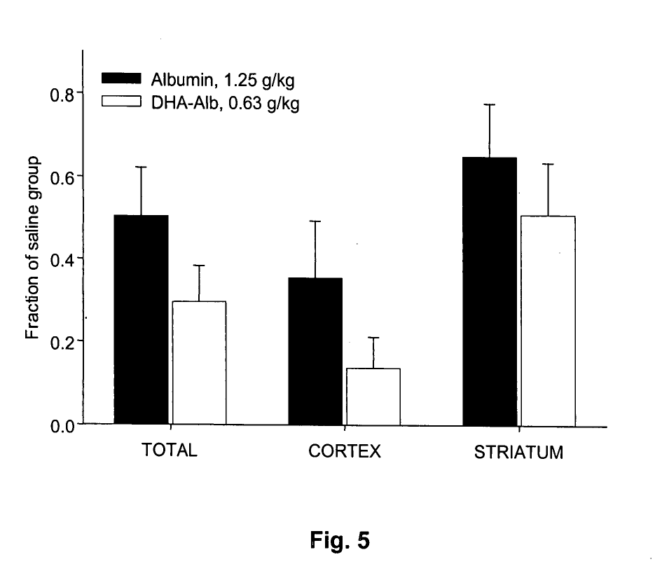

Finally, as shown in FIG. 5, the statistical comparison of DHA-Albumin, 0.63 g/kg treatment and Albumin, 1.25 g/kg treatment revealed no significant differences. This underscores the fact that DHA-Albumin, 0.63 g/kg treatment is as fully neuroprotective as treatment with albumin, 1.25 g/kg; in other words, when DHA is added to albumin, a high-grade neuroprotective effect is achieved at lower albumin doses.

In addition to reduced infarct areas, DHA-Albumin complex exhibited a dramatic decrease in brain swelling as estimated by the wet weight/dry weight method after cerebral injury. The following table illustrates the efficacy of DHA-Albumin complex to reduce brain swelling.

| | |
|---|---|
| Saline | 8.9 ± 5.2% |
| Albumin, 1.25 g/kg | 9.2 ± 3.9% |
| DHA-Albumin, 1.25 g/kg | 7.9 ± 4.0% |
| Albumin, 0.63 g/kg | 9.4 ± 7.0% |
| DHA-Albumin, 0.63 g/kg | 5.5 ± 2.4% |

* p = 0.004 vs. saline, Student t-test

Based on these results, the DHA-Albumin complex is an effective neuroprotective agent that can be used in treating ischemic stroke, injuries that may produce ischemic or traumatic tissue damage, and for reducing the potential for ischemic tissue damage during surgical procedures.

Although the present invention has been described in terms of specific embodiments, it is anticipated that alterations and modifications thereof will no doubt become apparent to those skilled in the art. It is therefore intended that the following claims be interpreted as covering all alterations and modifications that fall within the true spirit and scope of the invention.

What is claimed is:

1.-11. (canceled)

12. A method of inhibiting ischemic and hemorrhagic tissue damage comprising administering a therapeutically effective dose of a pharmaceutical composition comprising albumin and a polyunsaturated fatty acid.

13. The method of claim 12 wherein the albumin is human serum albumin.

14. The method of claim 12 wherein the albumin is recombinant human albumin.

15. The method of claim 12 wherein the polyunsaturated fatty acid is docosahexaenoic acid.

16. The method of claim 15 wherein the pharmaceutical composition is prepared by forming an albumin-polyunsaturated fatty acid complex by incubating albumin in the presence of docosahexaenoic acid such that the docosahexaenoic acid physically binds to the albumin forming an albumin-docosahexaenoic acid complex.

17. The method of claim 16 wherein the therapeutically effective dose comprises from about 0.25 grams per kilogram of bodyweight to about 2.5 grams per kilogram of bodyweight of the albumin-docosahexaenoic acid complex on an albumin weight basis.

18. The method of claim 16 wherein the therapeutically effective dose comprises from about 0.5 milligrams per kilogram of bodyweight to about 7.5 milligrams per kilogram of bodyweight of the albumin-docosahexaenoic acid complex on an docosahexaenoic acid weight basis.

19. The method of claim 12 wherein the ischemic and hemorrhagic tissue damage is caused by one or more of causes selected from the group comprising ischemic stroke, carotid endarterectomy, coronary artery bypass surgery, coronary angioplasty, thoracic or abdominal aortic reconstructions, mesenteric or renal vascular reconstruction, infrainguinal procedure, arterial stint surgery, venous surgery, major vascular trauma reconstruction, traumatic brain injury, traumatic eye injury, and traumatic spinal cord injury.
